(19) United States
(12) Patent Application Publication
(10) Pub. No.: US 2005/0070011 A1
(43) Pub. Date: Mar. 31, 2005

(54) Method and device for replicating arrays of cell colonies

(76) Inventors: Pawel Kuzan, Oakville (CA); Charles Boone, Toronto (CA)

Correspondence Address: Ralph A. Dowell of DOWELL & DOWELL P.C., 2111 Eisenhower Ave., Suite 406, Alexandria, VA, 22314, US

(21) Appl. No.: 10/673,545

(22) Filed: Sep. 30, 2003

(51) Int. Cl. C12Q 1/24; C12M 1/34

(52) U.S. Cl. 435/287.2

(57) ABSTRACT

The present invention is directed to a replicating pad adapted to be gripped by a replicating device. The replicating pad has a generally planar body and a plurality of pins extending downwardly from the body. All pins may have the same dimensions or different dimensions. In another aspect of the invention a replicating device is adapted to be used in association with a replicating pad having a plurality of pins extending downwardly. The replicating device includes a gripper, a method of aligning the replicating pad in the gripper and a method of pushing the replicating pad downwardly. The gripper is adapted to grip the replicating pad. In a further aspect of the invention a method of replicating cell colonies is disclosed.

### FIELD OF THE INVENTION

This invention relates to devices for manipulate arrays of cell colonies and in particular methods and devices that can manipulate large arrays of cell colonies.

### BACKGROUND OF THE INVENTION

Replicating devices are well known and are used to handle cell colonies and research associated therewith. Replicating devices are used in association with cell-based screens where many types of cells or colonies are exposed to a reagent (e.g. drug) to determine the sensitivity of the cells within the colony. They may also be used in association with cell-based screens where the source cells/colonies are mated or crossed or mixed with target cells, for instance the two-hybrid assay, yeast synthetic genetic array methodology as applied to synthetic genetic analysis or plasmid-based over-expression screens. They are used with cell-based screens where one or more types of cell are exposed to many types of compound, for instance a combinatorial library of chemicals, or a library of oligonucleotides that reduce gene transcript levels. Replicating devices are used in miniaturization of diagnostic applications where a clinical isolate is screened for drug sensitivity (e.g. bacterial strain replicated to array of different antibiotics) or for the presence of antigens (e.g. blood plasma sample replicated to array of antibodies). These devices may also be used for the curation, storage, mass production, and maintenance of biological libraries, arrays, clones, drugs, strains, clinical samples and other resources.

Defined cell arrays can be manipulated to facilitate genetic and proteomic applications on a large scale. Replicating devices allow researchers to combine different input colony arrays and to generate an output colony array containing positive events. Some of biological applications include use of the replicating device for analysis of protein-protein interactions with the yeast two-hybrid system [Utez et al., Nature 403: 601 (2000)], large-scale genetic analysis with the synthetic genetic array methodology [Tong et al., Science 294:2364 (2001)], chemical genetic drug sensitivity screens [Chang et al., Proc. Nail. Acad. Sci. 99: 16934-16939 (2002)]]. In principle, all types of liquid samples, or cells, (prokaryotic and eukaryotic, fungi, plant, and animal) or combinations there of, can be manipulated by the invention.

Today's state-of-the-art devices for replicating cell colony arrays use “bed-of-nails” print heads, where a large number of free floating metal pins are fitted into an array of holes in a metal plate manipulated by the robot. An example of such system is the CPCA (Colony Picker Colony Arrayer) robot from Bio-Rad. Replicating devices based on floating metal pins have two basic limitations. Firsty, as the number of pins in the replicating device increases, and, as a result, the diameter of the pins and spacing between individual pins decreases, the replicating device becomes increasingly difficult and costly to manufacture. Currently, the array of 1536 pins is considered the limit for practical applications. Secondly, after each transfer the pins need to be thoroughly washed to avoid cross-contamination of samples picked up by individual pins. In practice, the washing of pins takes several times longer than replicating the array transfer itself, which makes the entire process very inefficient.

Accordingly it would be advantageous to provide a replicating device that includes a large number of pins. In addition it would be advantageous to provide a replicating device and method of using same that reduces the time between replicating cell colonies.

### SUMMARY OF THE INVENTION

The present invention is directed to a replicating pad adapted to be gripped by a replicating device. The replicating pad has a generally planar body and a plurality of pins extending downwardly from the body. In one embodiment the pins all have the same dimensions. However, if desired, the pins may have different dimensions.

In another aspect of the invention a replicating device is adapted to be used in association with a replicating pad having a plurality of pins extending downwardly. The replicating device includes a gripper, a method of aligning the replicating pad in the gripper and a method of pushing the replicating pad downwardly. The gripper is adapted to grip the replicating pad.

In a further aspect of the invention a method of replicating cell colonies is disclosed. The method includes the steps of: picking up a replicating pad having a plurality of pins extending downwardly therefrom; lowering the replicating pad onto the cell colony; pressing the replicating pad into the cell colony such that the pins of the replicating pad engage the cell colony; lifting the replicating pad from the cell colony; lowering the replicating pad onto an agar plate; pressing the replicating pad into the agar plate such that the pins of the replicating pad engage the agar plate; removing the replicating pad from the agar plate; and releasing the replicating pad into a predetermined position.

The invention is particularly valuable for creating and manipulating high-density arrays. For many applications, there is an advantage of producing high density arrays because large numbers of colonies can be replicated in a single cycle of the robot, which would accelerate the pace of the project. For example, standard plastic plates filled with solid agar medium, which generate a ˜110 mm by ˜70 mm agar surface, are often used to grow the cell colonies. First, a series of low-density arrays is produced manually. Robotic equipment is then used to create higher density arrays by replicating a number of lower density arrays onto a single agar plate. Finally the high-density array is copied by replica-plating. The exact size or shape of the plate is not specific to the invention; indeed, one of the advantages of the invention is that specific arrays differing In size, density, and format are easily configured for a particular application.

Further features of the invention will be described or will become apparent in the course of the following detailed description.

### BRIEF DESCRIPTION OF THE DRAWINGS

The invention will now be described by way of example only, with reference to the accompanying drawings, in which:

FIG. 1 is cross-sectional view of a 768-pin replicating pad of the present invention and cell colonies deposited therewith;

FIG. 2 is a cross-sectional view of a 13,824-pin replicating pad of the sent invention and cell colonies deposited therewith;

FIG. 3 is a side view of a 768-pin replicating pad of the present invention;

FIG. 4 is an enlarged side view taken of FIG. 3;

FIG. 5 is a top view of a 768-pin replicating pad of FIG. 3;

FIG. 6 is an enlarged cross-sectional view taken along line 6-6 of FIG. 5;

FIG. 7 is a side view of a 13,824-pin replicating pad (partial pattern) of the present invention;

FIG. 8 is an enlarged side view taken of FIG. 7;

FIG. 9 is a top view of the 13,824-pin replication of FIG. 7;

FIG. 10 is a perspective view of the pad gripper constructed in accordance with the present invention;

FIG. 11 is a perspective view of the pad container constructed in accordance with the present invention; and

FIG. 12 is a perspective view of the pad locating device constructed in accordance with the present invention.

### DETAILED DESCRIPTION OF THE INVENTION

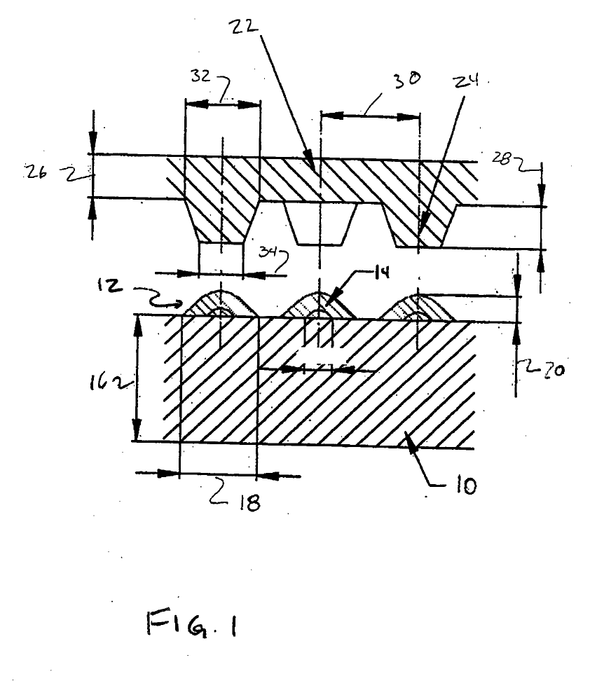

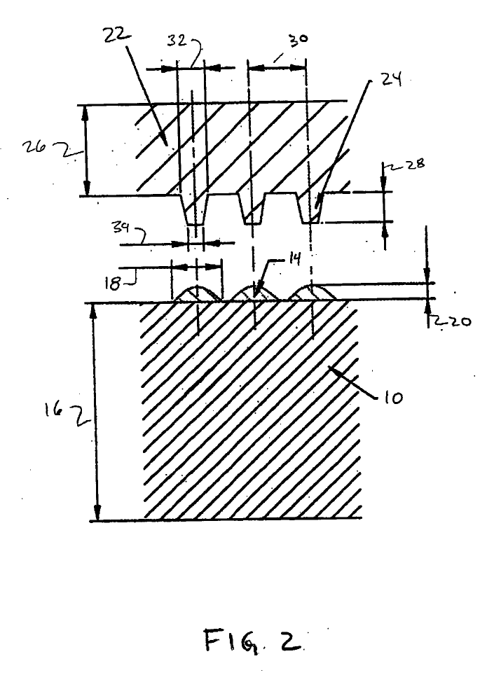

FIGS. 1 and 2 illustrate the replicating principle for 768- and 13,824-colony arrays, respectively. The theoretical limit of the print heads is dependent on the mold-building method. It is understood that the maximum density of the pins is likely higher than the density that would be practical for some biological manipulations. The application to a particular cell type will depend upon the growth characteristics of the cell type, i.e. rate of growth and colony shape and form. Alternatively the replicating device and method herein may also be used for liquid samples and as with the cell colonies the characteristics of the print head may be designed based on the characteristics of the liquid sample.

When growing on an agar surface 10, the yeast cell colonies 12 form small domes 14. Typically the agar surface is 3 mm thick as shown at 14 in FIGS. 1 and 2. The dimensions of each dome 14 are by way of example 1.75 mm or 0.65 mm in diameter 18 and 0.6 mm or 0.2 mm in height 20, respectively.

The replicating pad 22, shown above the agar surface 10, has a pattern of protrusions (pins) 24 matching the pattern of yeast cell colonies. When the pad 22 is lowered onto the agar surface 10, the pins 24 come in contact with their respective cell colonies 12 and pick up some of the sample. When the pad 22 is lowered onto another agar plate, some of the sample material is deposited on the agar surface 10 of the other plate, in an identical pattern. In the example shown in FIG. 1 the pad 22 has 768 pins 24. This pad has a pad thickness 26 of 1 mm and a pin 24 height 28 of 1 mm. The spacing 30 between the pins is 3.2 mm. The upper width 32 of the pin is 1.7 mm and the lower width 34 of the pin is 1 mm. Alternatively the example shown in FIG. 2 is a replicating pad 22 having 13,824 pins 24. In this example the pad thickness 26 is 1.4 mm and the pin height 28 is 0.4 mm. The spacing 30 between the pins is 0.75 mm. The upper width 32 of the pin is 0.6 mm and the lower width 34 of the pin is 0.3 mm. The replicating pad 22 shown in FIG. 2 corresponds to a yeast colony 12 having a plurality of small domes 14 with a height 20 of 0.2 mm and a diameter 18 of 0.65 mm. It will be appreciated by those skilled in the art that the number of pins 24 per replicating pad 22 can very greatly and that those shown in FIGS. 1 and 2 are by way of example only. For example the replicating pad could also have other multiples of 1536 namely 1536×4=6144, 1536×9=13824, 6144×4=24576, 6144×9=55296 etc.

Since the agar 10 is poured into the plastic plates as a liquid, the agar surface is essentially flat. However, for practical reasons agar thickness and its surface attitude (tilt) may vary slightly from plate to plate. Therefore, all pins 24 of a flat replicating pad 22 will come in contact with the agar surface, as long as the pad can be 10 adapted to varying height and tilt of the agar surface 10. This is described in more detail below.

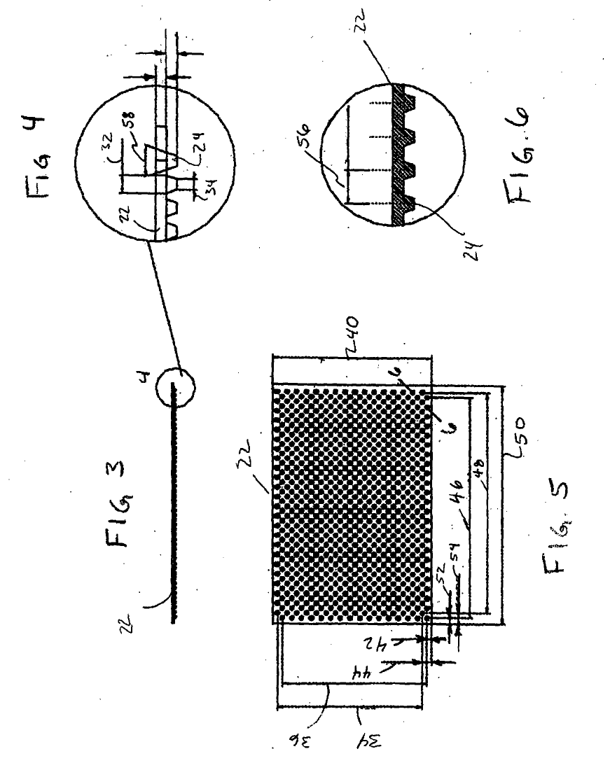

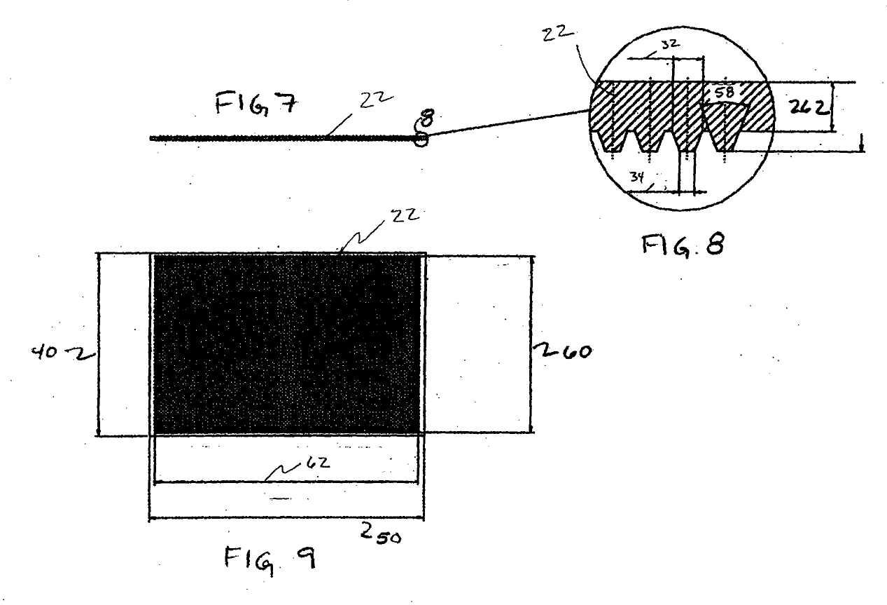

FIGS. 3, 4, 5 and 6 show the disposable pads 22 with a replicating pin density of 768 pins and correspond to the pad 22 shown in FIG. 1. It can be seen that there are sixteen (16) rows of pins 24 along the width of the pad as shown at 36. These are offset by a second set of sixteen (16) rows of pins shown at 34. If the pad 22 has a total width 40 of 74 mm there is a margin 42 of 2.12 mm between the edge and the closest pin and a margin 44 of 4.37 between the edge and the adjacent offset pin. Looking at the arrangement of the pins along the length of the pad there are twenty-four (24) pins in each row as shown at 46. These are offset by a second set of twenty-four (24) pins in each offset row as shown at 48. If the pad has a total length 50 of 112 mm, there is a margin 52 of 3 mm between the edge and the closest pin and a margin 54 of 5.25 between the edge and a adjacent offset pin. FIG. 6 shows the spacing of the pins 24 when viewed along the diagonal. Specifically the angled spacing 56 is 3.2 mm. FIG. 4 also shows the angle 58 of pin 24 which is 39°.

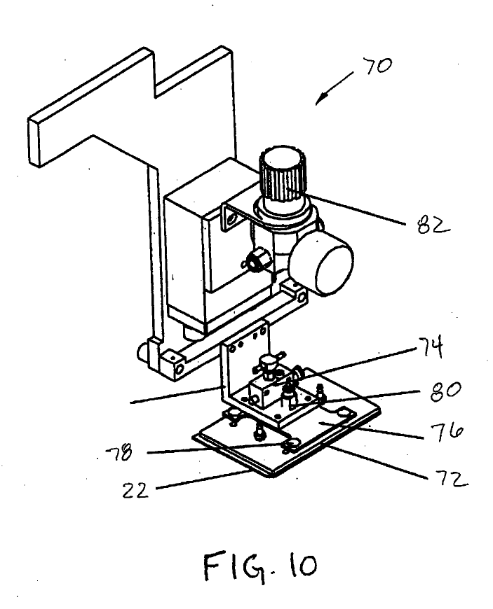

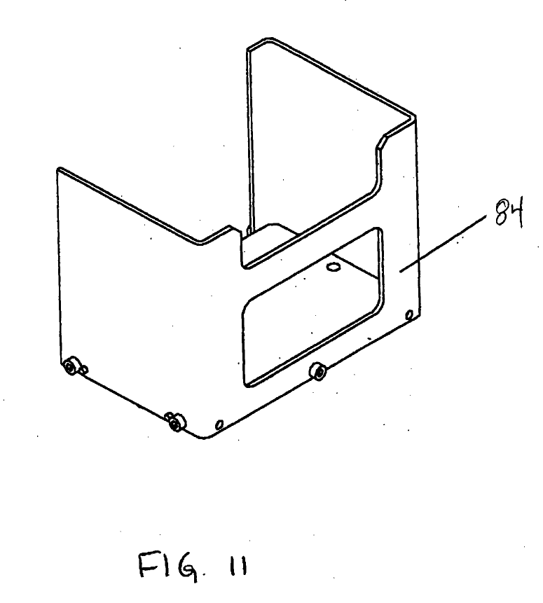

Similarly FIGS. 7, 8 and 9 show the disposable pad 22 with a replicating pin density of 13,824, which corresponds to FIG. 2. As discussed above the pad 22 shown in FIGS. 2, 7, 8 and 9 does not include pins that are offset. This embodiment shows ninety-six (96) rows of pins along the width as shown at 60. As above the total width 40 of the pad 22 is 74 mm. This embodiment shows one hundred and forty-four (144) pins 24 in each row along the length as shown at 62. As above the total length 50 is 112 mm. FIG. 8 shows the angle 58 of pin 24 as 41°. In the preferred embodiment the replicating pads 22 are injection molded from an inexpensive material, such as polystyrene. Replicating pads 22 with other pin densities and patterns can be produced using the same manufacturing techniques. A set of pads 22 would be required for producing higher density arrays from lower density arrays, and for replicating high-density arrays. Preferably the pin diameter corresponds with the colony size of the highest density being handled by the particular pad, such that the colonies in the arrays being built do not overlap. A series of identical pads with lower-density small-diameter pins may be used to create higher-density patterns. For each subsequent transfer the pad would be offset, such that the new colonies are printed in-between the previously printed colonies. Accordingly it may be possible to build a 1,536 array from a series of 96 arrays (16×increase), or an intermediate 384 array needs to be created (4×increase twice).

FIG. 10 shows the replicating device or pad gripper generally at 70. The replicating device is adapted to be attached to a robot (not shown). Preferably vacuum is used to attach replicating pad 22 to bottom plate 72 of the gripper 70. In this embodiment vacuum is produced by a small vacuum generator 74, although it could also be supplied by an external vacuum pump. The bottom plate 72 is attached to the gripper plate 76 with four conical pins 78 protruding through their corresponding holes in gripper plate 76. When the gripper is above the agar surface (not shown), conical pins 78 accurately locate bottom plate 72 with respect to gripper plate 76. When the gripper is lowered toward the agar surface, bottom plate 72 with replicating pad 22 attached thereto rests on the agar surface, while conical pins 78 separate from their respective holes in gripper plate 76. This configuration allows the gripper to accommodate, to a certain degree, uncertain height and slight tilt of the agar surface.

A small pneumatic actuator 80 attached to gripper plate 76 is used to press down at the center of gripper plate 76. When bottom plate 72 with replicating pad 22 attached thereto rests on the surface of an agar plate, the actuator 80 is activated to assure positive contact between all pins and their corresponding cell colonies. Pressure regulator 82 is used to adjust the force that the actuator 80 exerts on bottom plate 72.

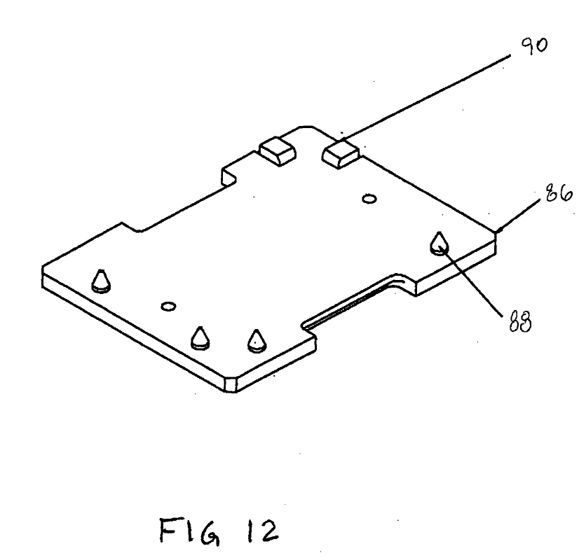

FIG. 11 shows the open top container 84, which stores a stack of disposable replicating pads (not shown in FIG. 11). The gripper picks up the pads 22 from container 84. Since positioning of the pads in container 84 is not accurate, a separate pad-locating plate or adapter 86, shown in FIG. 12, is mounted on the robot platen next to container 84. Conical locating pins 88 and blocks 90 are used to accurately position the replicating pad with respect to the robot workspace.

In operation, the robot lowers the gripper 70 into the pad container 84 where vacuum is used to attach a replicating pad 22 to the bottom of gripper plate 76. The pad is then transferred to the pad-locating adapter 86 and released just above the adapter surface. While falling into the adapter 86, the replicating pad is accurately positioned by pins 88 and blocks 90. The gripper again picks up the pad from adapter 86 and carries it over to the first agar plate. With actuator 80 released, the gripper 70 moves toward the agar plate until pad 22 rests on the agar surface 10. At this point, actuator 80 is momentarily activated to assure full contact between the pins 24 of the replicating pad 22 and their corresponding cell colonies 12. The gripper 70 then moves over to the second agar plate and lowers the pad 22 onto the agar surface 10 in an identical manner. Once the colony array transfer is completed, the gripper moves over to a waste container (not shown) and the replicating pad is released into this container. Alternatively the replicating pad is released into a storage container and the replicating pad is washed thereafter. The replicating pad may be washed individually or in bulk to be recycled and reused. The entire replicating cycle as described above is then repeated as required.

It will be appreciated by those skilled in the art that a mechanical gripper rather than a vacuum gripper could be used to hold the replicating pad. The pad container or the replicating pad could be modified to eliminate the pad positioning attachment. There are many alternate arrangements that could be used to press down on the replicating pad for example a spring could be used. The system could be modified so that rather than compliant mounting of the replicating pad at the gripper compliance is provided at the agar plate.

As used herein, the terms “comprises” and “comprising” are to be construed as being inclusive and opened rather than exclusive. Specifically, when used in this specification including the claims, the terms “comprises” and “comprising” and variations thereof mean that the specified features, steps or components are included. The terms are not to be interpreted to exclude the presence of other features, steps or components.

It will be appreciated that the above description related to the invention by way of example only. Many variations on the invention will be obvious to those skilled in the art and such obvious variations are within the scope of the invention as described herein whether or not expressly described.

1. A replicating pad adapted to be gripped by a replicating device comprising:
a generally planar body; and
a plurality of pins extending downwardly from the body.

2. A replicating pad as claimed in claim 1 wherein all of the plurality of pins have generally the same dimensions.

3. A replicating pad as claimed in claim 2 wherein the pins are arranged in a plurality of rows and each row has a plurality of pins.

4. A replicating pad as claimed in claim 3 wherein the pins are arranged to correspond with one of a cell colony and a liquid sample.

5. A replicating pad as claimed in claim 3 wherein there are 768 pins.

6. A replicating pad as claimed in claim 3 wherein there are 1536 pins.

7. A replicating pad as claimed in claim 3 wherein there are 13,824 pins.

8. A replicating pad as claimed in claim 3 wherein there are 2144 pins.

9. A replicating pad as claimed in claim 3 wherein there are 24576 pins.

10. A replicating pad as claimed in claim 3 wherein the replicating pad is constructed by way of injection molding.

11. A replicating pad as claimed in claim 10 wherein the replicating pad is constructed from polystyrene.

12. A replicating pad as claimed in claim 3 wherein the replicating pad is designed to be used once and then disposed of.

13. A replicating pad as claimed in claim 3 wherein the replicating pad is washed for reuse.

14. A replicating pad as claimed in claim 3 wherein the planar body is 112 mm by 74 mm.

15. A replicating device adapted to be used in association with a replicating pad having a plurality of pins extending downwardly, the replicating device comprising:
a gripper adapted to grip the replicating pad;
a means for aligning the replicating pad in the gripper; and
a means for pushing the replicating pad downwardly.

16. A replicating device as claimed in claim 15 wherein the pushing means includes an actuator and a pressure regulator.

17. A replicating device as claimed in claim 16 further including a pad locating device.

18. A replicating device as claimed in claim 17 wherein the pad locating device has a plurality of projections extending upwardly therefrom.

19. A replicating device as claimed in claim 18 wherein the projections include conical pins and blocks.

20. A replicating device as claimed in claim 19 further including a pad container adapted to hold a plurality of replicating pads.

21. A replicating device as claimed in claim 15 wherein the gripper includes a vacuum to hold the replicating pad to the gripper.

22. A method of replicating cell colonies comprising the steps of:
picking up a replicating pad having a plurality of pins extending downwardly therefrom;
lowering the replicating pad onto a cell colony;
pressing the replicating pad into the cell colony such that the pins of the replicating pad engage the cell colony;
lifting the replicating pad from the cell colony;
lowering the replicating pad onto an agar plate;
pressing the replicating pad into the agar plate such that the pins of the replicating pad engage the agar plate;
removing the replicating pad from the agar plate; and
releasing the replicating pad into a predetermined position.

23. A method of replicating cell colonies as claimed in claim 22 further including the step of aligning the replicating pad in a gripper.

24. A method of replicating cell colonies as claimed in claim 23 further including the step of aligning the replicating pad with the agar plate.

25. A method of replicating cell colonies as claimed in claim 24 wherein the replicating pad is released into a disposal unit.

26. A method of replicating cell colonies as claimed in claim 22 wherein the replicating pad is a first replicating pad and the replicating pad is lowered at a defined location and further including a step of providing a second replicating pad and repeating the steps with a second replicating pad and wherein the second replication pad is lowered at a location offset from the defined location.
